(19) United States
(12) Patent Application Publication
(10) Pub. No.: US 2008/0269769 A1
(43) Pub. Date: Oct. 30, 2008

(54) SYSTEM AND METHOD FOR PREPARING A CORNEAL GRAFT

(75) Inventor: Alexander Dybbs, Cleveland, OH (US)

Correspondence Address: RENNER OTTO BOISSELLE & SKLAR, LLP, 1621 EUCLID AVENUE, NINETEENTH FLOOR, CLEVELAND, OH, 44115, US

(73) Assignee: AZD HOLDING, LLC, Beachwood, OH (US)

(21) Appl. No.: 12/111,097

(22) Filed: Apr. 28, 2008

Related U.S. Application Data
(60) Provisional application No. 60/914,529, filed on Apr. 27, 2007.

(51) Int. Cl. A61F 9/007

(52) U.S. Cl. 606/107; 606/166

(57) ABSTRACT

A system (100) and method for preparing a corneal graft from a corneo-scleral button for keratoplasty, such as DLEK, DSEK, or DSAEK, are characterized by the combination of a sterile, disposable, artificial anterior chamber (102) for holding a corneo-scleral button, and a sterile, disposable keratome (104, 130) having a cutting blade (136) for cutting a corneal flap or resection from the button. The keratome (104, 130) and the artificial chamber (102) are substantially entirely plastic and disposable, and a reusable control assembly (106, 400) is connected to the keratome (104, 130) by a control cable (402) to automatically cut the button. The control cable (402) includes a shaft (406) movable within a sheath (404). The artificial chamber (102) includes a stand (112) for supporting a corneo-scleral button, and a cap (114, 302) mountable to the stand (112) to hold the button in place while it is cut. The keratome (104, 130) includes a base (132) and a handle (342), either or both of which is integral with or attachable to the cap (114, 302).

This application claims the benefit of U.S. Provisional Patent Application No. 60/914,529 filed Apr. 27, 2007, which is incorporated herein in its entirety.

### FIELD OF THE INVENTION

The present invention relates to a system and method for preparing a corneal graft, particularly a system including an artificial anterior chamber and/or a keratome and related methods.

### BACKGROUND

Traditionally corneal transplants have involved the complete replacement of the full thickness of the cornea. Recently new procedures have been developed that replace only inner layers of the cornea, thereby generally providing improved vision with fewer complications. These procedures include various types of posterior lamellar keratoplasty, such as deep lamellar endothelial keratoplasty (DLEK), Descemet's stripping with endothelial keratoplasty (DSEK), and Descemet's stripping automated endothelial keratoplasty (DSAEK).

In these procedures a donor disk with a thickness of about 150 microns, generally encompassing layers of stroma to endothelium, is cut from a donor corneal/scleral button with a corneal thickness of about 550 microns. Following recent advances in conservation fluids, the donor cornea can be conserved for a few days until the implantable disk is prepared and then inserted in the diseased or otherwise impaired cornea.

This button consists of a cornea and a ring of sclera bounding the cornea. The implantable donor disk is prepared from a donor corneal/scleral button using an assembly known as an artificial anterior chamber. The artificial chamber holds the donor button by clamping the scleral ring while supporting the corneal portion. A technician or surgeon cuts a section from the upper surface of the donor button, and then a surgeon uses a punch to obtain the disk that is then implanted. Often the section is cut from the donor button by technicians at eye banks and then the section is replaced and the cornea is preserved in a fluid. This preserved tissue is then sent to surgeons who complete the preparation and implantation of the disk. If the same chamber is used again, the artificial chamber must be thoroughly cleaned and sterilized before another corneo-scleral button can be mounted in the chamber.

### SUMMARY

The present invention improves on prior corneal/stromal button preparation, storage, and/or transport systems that include artificial chambers by providing an improved and disposable artificial chamber, and a combination of a disposable artificial chamber and a disposable keratome, while also improving the quality of the corneal resection by automating the cutting process.

A system and method for preparing a corneal graft from a corneo-scleral button for keratoplasty, such as DLEK, DSEK, or DSAEK, are characterized by the combination of a sterile, disposable, artificial anterior chamber for holding a corneo-scleral button, and a sterile, disposable keratome having a cutting blade for cutting a corneal flap or resection from the button. The keratome and the artificial chamber are substantially entirely plastic and disposable, and a reusable control assembly is connected to the keratome by a control cable to automatically cut the button. The control cable includes a shaft movable within a sheath. The artificial chamber includes a stand for supporting a corneo-scleral button, and a cap mountable to the stand to hold the button in place while it is cut. The keratome includes a base and a handle, either or both of which is integral with or attachable to the cap.

In particular, the present invention provides:

An artificial anterior chamber for preparing a corneal graft from a corneo-scleral button that comprises (A) a stand having a support pedestal to support a donor corneo-scleral button at a distal end, (B) a cap mountable over the support pedestal, and (C) a coupling collar for securing the cap to the base without rotating the cap relative to the base.

An artificial anterior chamber for preparing a corneal graft from a corneo-scleral button that comprises (A) a stand having a support pedestal to support a donor corneo-scleral button at a distal end, and (B) a cap mountable over the distal end of the support pedestal. And the stand and the cap include cooperating anti-rotation elements that prevent relative rotation between the cap and the stand.

An artificial anterior chamber for preparing a corneal graft from a corneo-scleral button that comprises (A) a stand having a support pedestal to support a donor corneo-scleral button at a distal end, and (B) a cap mountable over the distal end of the support pedestal. And the stand and the cap include opposed generally parallel surfaces that cooperate to clamp a portion of the corneo-scleral button therebetween.

An artificial anterior chamber for preparing a corneal graft from a corneo-scleral button that comprises (A) a stand having a support pedestal to support a donor corneo-scleral button at a distal end, and (B) a cap mountable over the distal end of the support pedestal so that an opening in a top of the cap provides access to a corneo-scleral button supported on the pedestal. And the stand includes a fluid channel that terminates at a top of the pedestal to provide fluid support for a corneo-scleral button mounted thereon.

A method of preparing a corneal graft from a corneo-scleral button that comprises the steps of (A) mounting a corneo-scleral button in an artificial anterior chamber, (B) connecting a keratome to a remotely located control assembly, (C) controlling the keratome to cut a corneal portion of the button held in the artificial chamber, (D) disconnecting the keratome from the controller, and (E) disposing of the artificial chamber and the keratome.

A system for preparing a corneal graft that comprises a sterile, disposable artificial anterior chamber for holding a corneo-scleral button, and a sterile, disposable keratome having a cutting blade for cutting a corneal portion of the button.

A system for preparing a corneal graft from a corneo-scleral button comprises a stand having a support pedestal to support a donor corneo-scleral button at a distal end, and a keratome having a base, a carriage mounted to the base, and a cutting blade carried in the carriage. The carriage is guided for movement in a cutting direction relative to the base, and the base is configured for connection to a control cable having a sheath and a control shaft movable within the sheath. The base includes an anchor for fixing the sheath relative to the base such that axial movement of the control shaft effects movement of the carriage in the cutting direction. The base of the keratome is an integral part of a top of a cap, and the cap is mountable to the stand to define a cavity between the distal end of the pedestal and the cap to receive and hold a corneo-scleral button.

An artificial anterior chamber for preparing a corneal graft from a corneo-scleral button that comprises (A) a base having a support pedestal to support a donor corneo-scleral button at a distal end; (B) a cap mountable over the support pedestal, the cap having a support portion that fits over at least a portion of the support pedestal, a flange toward a bottom end of the support portion that extends beyond an outer extent of the support portion, and a keratome base toward a top end of the support portion that extends beyond a laterally outer extent of the support portion; and (C) a coupling collar for securing the cap to the base without rotating the cap relative to the base, the coupling collar substantially encircling the support portion of the cap, the flange and the platform cooperating to capture the coupling collar on the cap.

A method of assembling an artificial chamber to prepare a corneal graft from a corneo-scleral button that comprises the steps of: anchoring a keratome to an upper end of a cap, placing the cap over a pedestal portion of a stand, and coupling the cap to the stand without rotating the cap relative to the pedestal. The anchoring step includes attaching a keratome to the cap by anchoring a sheath to the base of the keratome. The keratome includes a carriage that is movable across the base, a cutting blade carried by the carriage, and a drive shaft connected to the carriage and extending through the sheath, whereby axial movement of the drive shaft effects linear movement of the carriage in a cutting direction that moves the cutting blade across the opening.

A method of preparing a corneal graft that comprises the following steps:

(A) placing a corneo-scleral button on a pedestal portion of a stand;

(B) placing a cap over the corneo-scleral button and the pedestal so that a portion of the cornea is visible through an opening in a base at the upper end of the cap;

(C) securing the cap to the stand with a coupling collar that engages a coupling portion of the stand to hold the cap in place relative to the stand without rotating the cap relative to the stand;

(D) supplying a fluid through a channel in the pedestal to force the cornea through the opening in the base;

(E) mounting a keratome to the base;

(F) connecting a control system to a keratome; and

(G) moving a cutting blade portion of the keratome across the base and the opening to cut the cornea.

The foregoing and other features of the invention are more fully described and particularly pointed out in the claims. The following description and annexed drawings set forth in detail two illustrative embodiments, these embodiments being indicative of various ways in which the principles of the invention may be employed.

### BRIEF DESCRIPTION OF THE DRAWINGS

FIG. 1 is a schematic diagram of a system provided by the present invention.

FIG. 2 is a perspective view from above of an exemplary combination of a keratome and an artificial anterior chamber provided by the present invention.

FIG. 3 is a perspective view from below of the combination shown in FIG. 2.

FIG. 4 is an exploded view of the combination shown in FIG. 2.

FIG. 5 is another exploded view of the combination shown in FIG. 2.

FIG. 6 is a perspective view from above of another exemplary combination of a keratome and an artificial anterior chamber provided by the present invention.

FIG. 7 is a perspective view from below of the combination shown in FIG. 6.

FIG. 8 is an exploded view of the combination shown in FIG. 6.

FIG. 9 is another exploded view of the combination shown in FIG. 6.

FIG. 10 is a top view of the combination shown in FIG. 6.

FIG. 11 is a front cross-sectional view of the combination as seen along lines 11-11 of FIG. 10.

FIG. 12 is a side/front cross-sectional view of the combination as seen along lines 12-12 of FIG. 10.

FIG. 13 is a side cross-sectional view of the combination as seen along lines 13-13 of FIG. 10.

FIG. 14 is a side view of a proximal portion of a keratome portion of the combination of FIG. 2.

FIG. 15 is an enlarged cross-sectional view of a portion of the keratome of FIG. 14.

FIGS. 16A-16D are sequential views illustrating sequential steps in connecting a keratome to a control assembly in accordance with the present invention.

FIG. 17 is a perspective view of a diagrammatic control system provided in accordance with the present invention.

FIG. 18 is an enlarged view of a control assembly portion of the control system of FIG. 17.

### DETAILED DESCRIPTION

### System

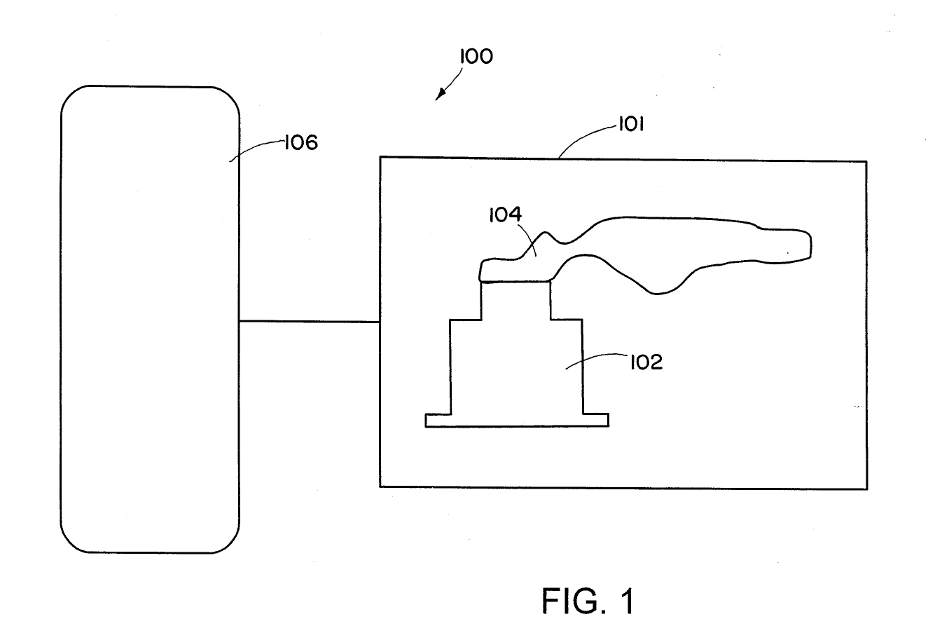

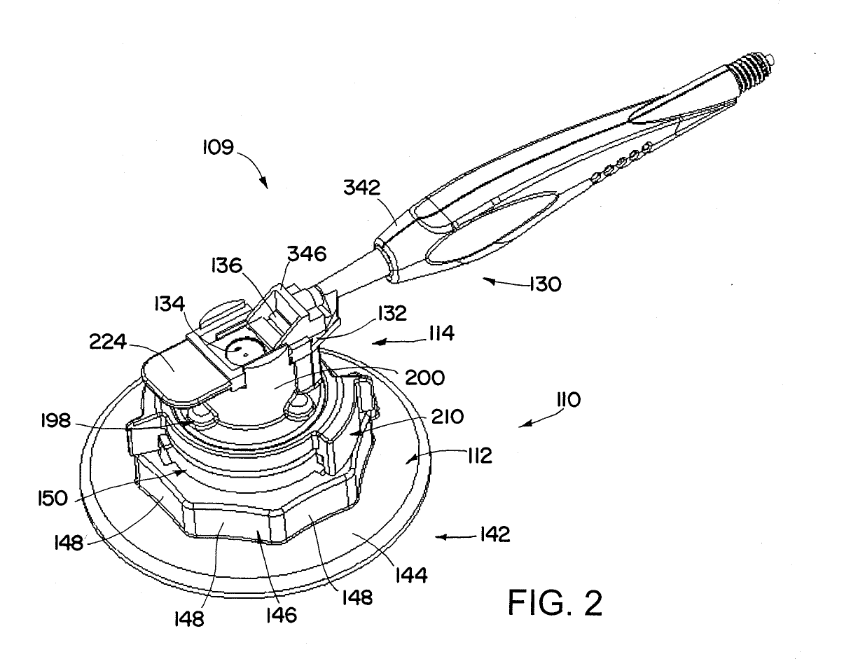

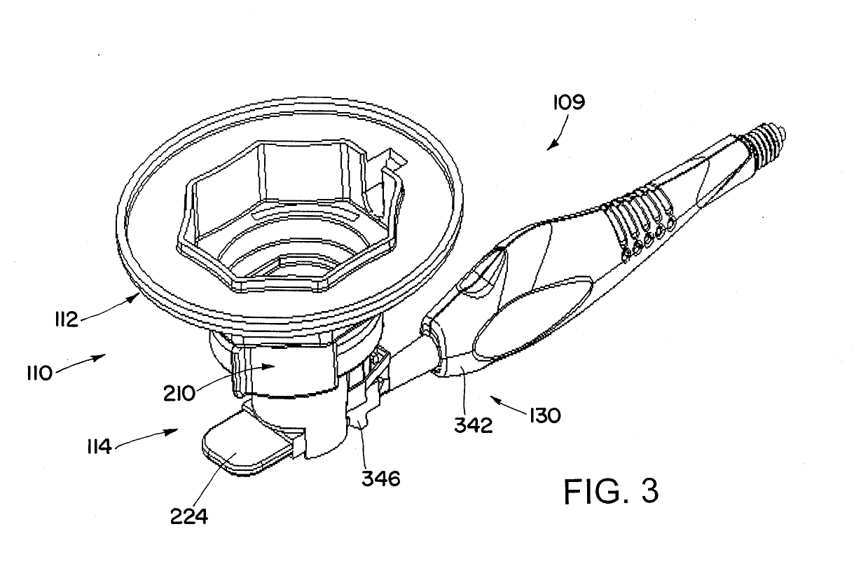

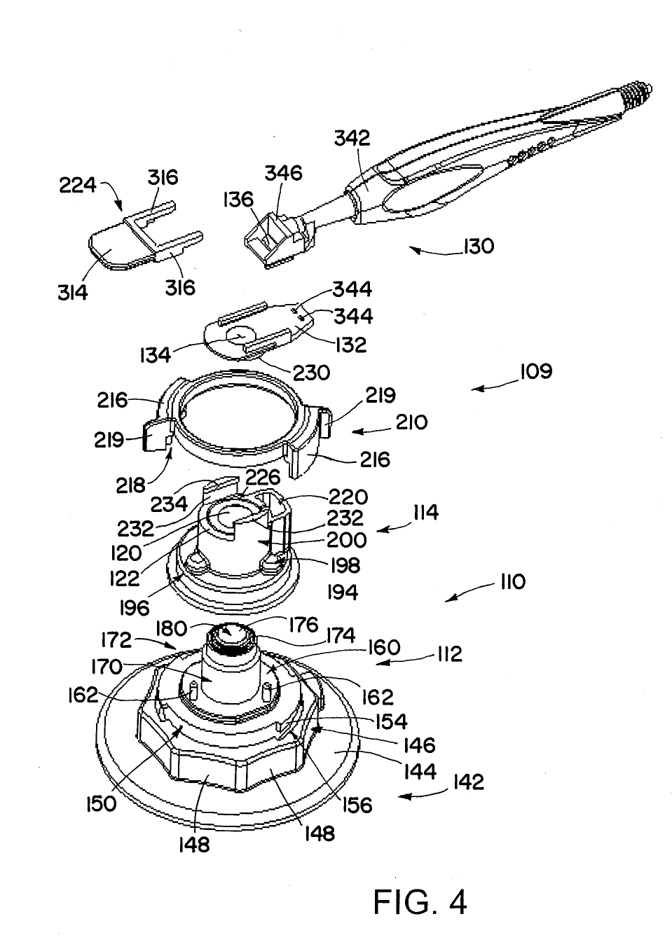

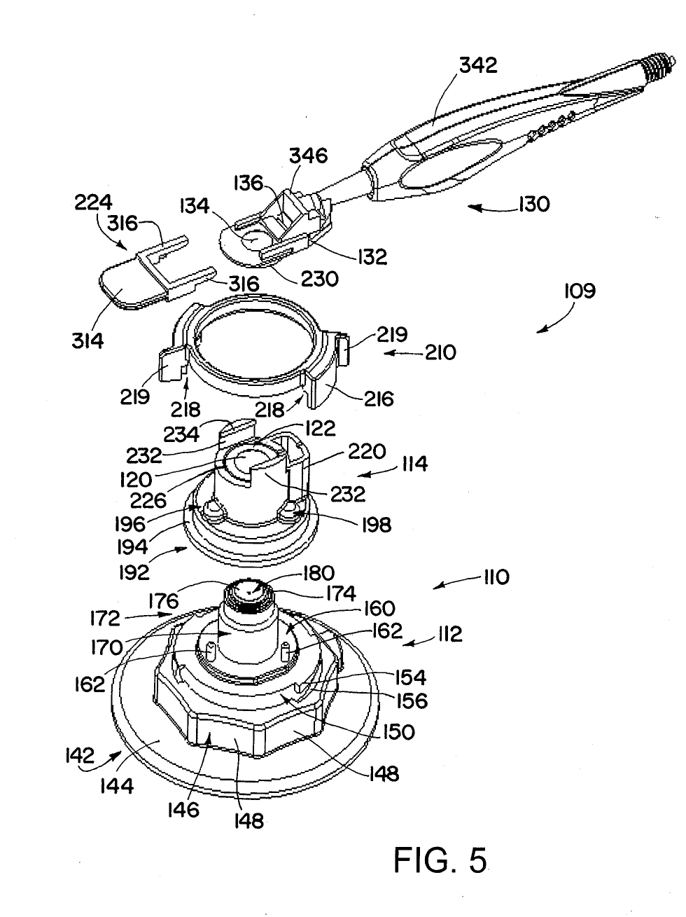

Referring now to the drawings in detail, FIG. 1 illustrates an exemplary system 100 provided by the invention for preparing a corneal disk from a corneo-scleral button. This system 100 includes the combination 101 of a disposable artificial anterior chamber 102 for supporting the corneo-scleral button, a disposable keratome 104 mountable to or integral with the artificial chamber 102 for cutting a flap or cap section from the button, and a remotely located and reusable control assembly 106 connected to the combination 101 for controlling the artificial chamber 102 and the keratome 104. The invention also provides a corresponding method. An exemplary combination 109 of an artificial chamber and a keratome is shown in FIGS. 2-5.

### Artificial Chamber

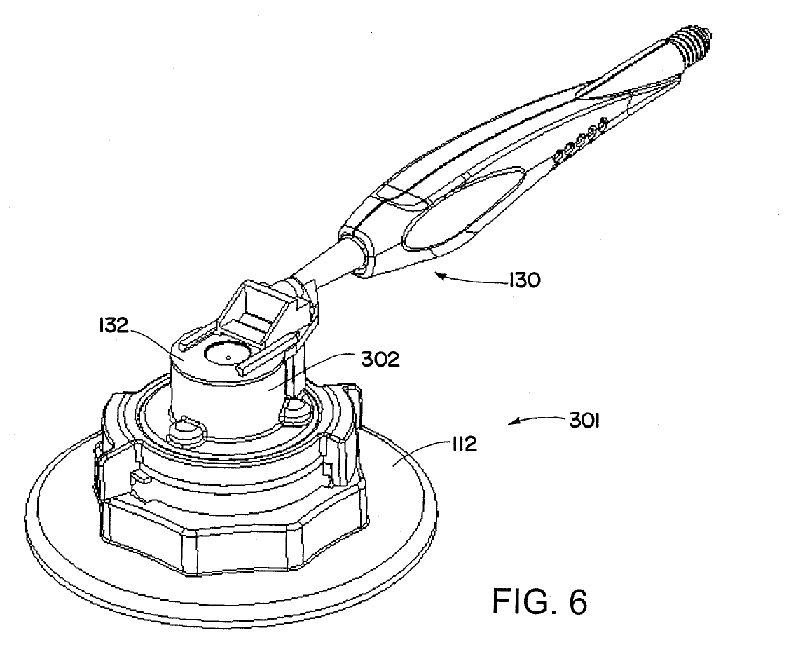

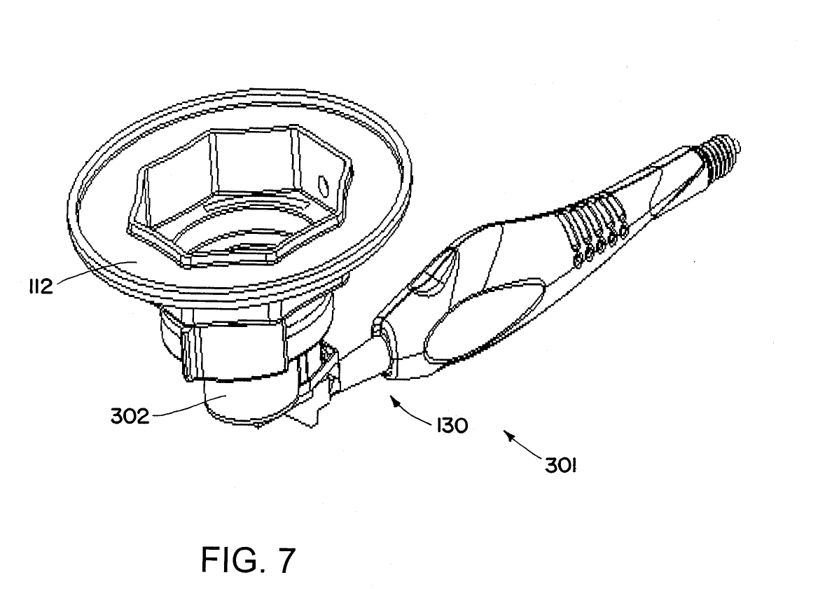

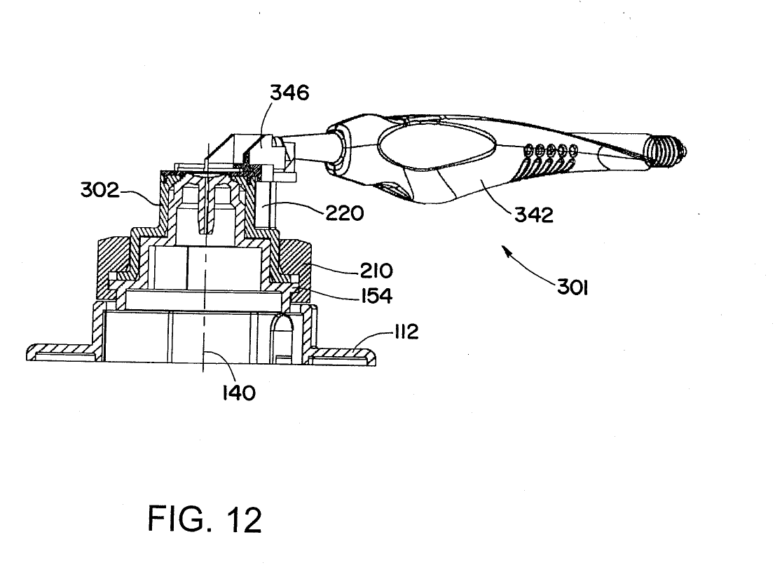

An exemplary artificial chamber 110 provided by the invention generally includes a stand 112 for supporting the corneo-scleral button and a cap 114 (generally corresponding to cap 302 in FIGS. 6-9 and 11-13 except as otherwise shown and described) mountable to the stand 112 to hold the disk in place between the stand 112 and the cap 114. The cap 114 and the stand 112 cooperate to define a cavity at an upper end of the stand 112 in which the corneo-scleral button is received. Within the cavity, a space behind the corneo-scleral button is filled with fluid to simulate the pressure in an anterior chamber of an eye and push the cornea upward away from the top of the stand 112 and toward an opening 120 at a top side 122 of the cap 114 that provides access to the cornea.

An exemplary keratome 130, meanwhile, generally includes a base 132 having an opening 134 therein that can be aligned with the opening 120 in the cap 114, and a cutting blade 136 that is supported and guided for movement in a cutting direction relative to the base 112 for cutting the flap or cap section from the corneo-scleral button. Although an exemplary keratome 130 is shown in the drawings, other keratomes can be used in accordance with the invention. For example, the illustrated keratome 130 cuts in a straight line, along a linear path. Some keratomes, however, rotate a cutting blade about an axis, cutting along a curved path. Either type of keratome can be used in the system provided by the invention.

### Stand

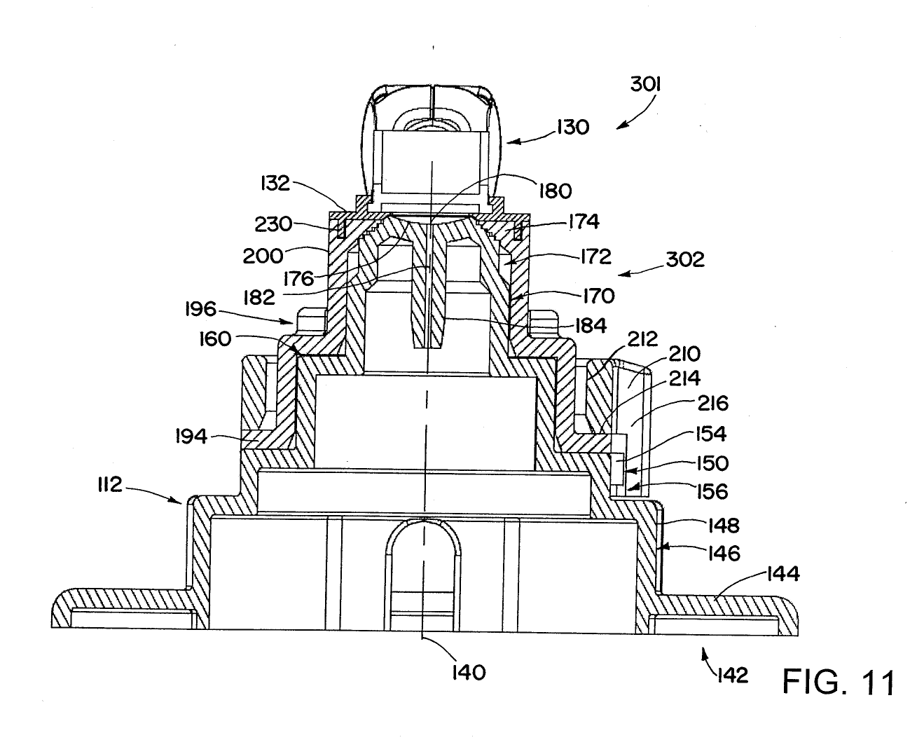

In the illustrated embodiments, the stand 112 is in the form of a stepped cylinder with a longitudinal axis 140 (FIG. 11) that generally is vertical. At a bottom side 142 the stand 112 includes a base flange 144 that helps a technician or surgeon hold the artificial chamber 110 on a horizontal work surface, such as a table. Rising from and inwardly stepped from the flange 144 is a faceted base portion 146. In the illustrated embodiment the base portion 146 has eight sides 148 that provide an octagonal cross-section. The faceted sides 148 of the base portion 146 help the technician hold onto the artificial chamber 110. The base portion 146 is not limited to an octagonal shape, equal sides, or eight sides.

Above and inwardly-stepped from the faceted base portion 146 is a generally cylindrical coupling portion 150 having coupling elements that help couple the cap 114 to the stand 112. In the illustrated embodiment the coupling elements include radially outwardly facing and outwardly extending protrusions 154 that define helical channels 156, similar to screw threads.

Further above and inwardly-stepped from the coupling portion 150 is a platform portion 160 having one or more anti-rotation elements that cooperate with corresponding elements in the cap 114 to minimize or prevent rotation of the cap 114 relative to the stand 112. In this embodiment, the anti-rotation elements include one or more pins 162, specifically three in the illustrated embodiment, radially spaced from and extending parallel to the longitudinal axis 140. The pins 162 are equally spaced about the circumference of the stand 112 in this embodiment.

The stand 112 also includes a support pedestal 170 inwardly stepped from the platform portion 160 that supports a donor corneo-scleral button at a distal and upper end 172 of the pedestal 170. The pedestal portion 170, like the other elements of the stand 112, typically is integral with the stand and is in a fixed position relative to the rest of the stand.

The upper end 172 of the pedestal portion 170 is further inwardly stepped and the top face further includes a frustroconical peripheral portion 174 and a concave or recessed central portion 176. The peripheral portion 174 has a plurality of concentric ridges that cooperate with corresponding ridges on a facing surface of the cap 114 to help grip and hold the donor disk in place. The central concave portion 176 has an outlet opening 180 for a fluid channel 182 to provide fluid under the corneo-scleral button.

The fluid channel 182 generally passes through the pedestal portion 170 and out the faceted base portion 146, typically to one side of the base portion. The fluid channel 182 may be integrally formed in the stand 112, or may be partially or wholly defined by a length of tubing. Silicon is an exemplary tubing material. In the illustrated stand 112 the fluid channel is defined in part by an integral fitting 184 extending from the outlet 180. The fitting 184 includes a tapered outer diameter for connection to a length of tubing that passes through a side wall 148 of the faceted base 146.

The tubing, connected to the fitting or an additional length of tubing, connects the fluid channel to a manually-actuated syringe or a pneumatic or hydraulic pump and a source of fluid. The tubing also can include or be connected to a filter to ensure that the fluid is sterile when it reaches the outlet 180. The fluid stiffens the cornea by pressurizing it from underneath, thus making it possible to cut the corneal button under conditions that approximate conditions that typically exist in an eye. The fluid includes a sterile fluid, for example a gas, such as air, or a liquid, such as Optisol™ corneal storage medium (Bausch & Lomb, Rochester, N.Y., US, formerly available from Chiron Ophthalmics, Irvine, Calif. US).

The pump and the source of fluid may be incorporated into the control assembly or may be separate therefrom. The pump or the syringe provides fluid through the channel 182 to the outlet 180 and the underside of a corneo-scleral button. The fluid pumped through the fluid channel 182 simulates the internal pressure of an eye and causes the disk to take on a convex shape similar to that of an eye. This helps to ensure that the cornea will protrude through the opening 120 in the top of the cap 114. The fluid also supports the cornea as it is being cut.

### Cap

The cap 114 mounts over the stand 112 to hold the corneo-scleral button on the stand 112. To mate with the stand 112, the illustrated cap 114 has an internal cavity with a stepped cylindrical shape along an axis that is aligned with the axis 140 of the stand 112 in FIG. 6. When installed, the opening 120 in the top end of the cap 114 generally is aligned with the distal end of the support pedestal 170 and a corneo-scleral button supported thereon.

The cap 114 has a bottom end 192 opposite the top end 122 that is open and fits over the pedestal portion 170 of the stand 112. The cap 114 has a flange 194 at the lower end 192 that seats on an upper surface of the coupling portion 150 of the stand 112. Inwardly stepped from the flange 194 is a registration section 196 with one or more anti-rotation elements, in this case three holes 198 for receipt of the corresponding anti-rotation pins 162 on the stand 112. In the illustrated embodiment, the holes are closed at the top end, but they could be open. Other arrangements are contemplated within the scope of the present invention, including other types or arrangements of anti-rotation elements. The pins and holes may be reversed relative to the cap and the stand or mixed, the spacing between the holes may be uniform or irregular, and more or fewer anti-rotation elements may be employed to good effect.

With three anti-rotation elements, the cap 114 must be rotated no more than sixty degrees to align a hole and a pin, and with equidistant pins and holes the rotational orientation of the cap 114 relative to the stand 112 generally is not significant. Additionally or alternatively, the artificial chamber 110 can include a flat surface or a protrusion and corresponding surface or slot in the cap 114 so that the cap 114 can only be put on the stand 112 one way.

Inwardly stepped and rising above the registration section 196 is a keratome support section 200. The keratome support section 200 is generally cylindrical and includes the opening 120 at the top end 122 of the cap 114. The keratome support section 200 receives the pedestal portion 170 of the stand 112 therein and cooperates with the pedestal 170 to define the cavity in which the corneo-scleral button is held. Inside the cap 114 and adjacent the opening 120, the cap 114 has a frustroconical surface with a plurality of ridges, ribs, beads, or stepped faces that protrude from the surface. This surface cooperates with the corresponding surface 174 at the upper end 172 of the pedestal portion 170 of the stand 112 to grip and hold a corneo-scleral button in a desired position therebetween, with the cornea accessible via the opening 120 at the top end 122 of the cap 114. This surface on the cap generally is parallel to the corresponding surface 174 on the pedestal portion 170. The ridges or other protrusions form annular rings that not only help to grip the corneo-scleral button but also cooperate with the corneo-scleral button to effect one or more annular line seals. These seals improve the ability of the fluid to provide uniform pressure to the back side of the corneo-scleral button, the side of the button that faces the outlet 180 of the fluid channel 182 at the top of the pedestal portion 170 of the stand 112.

Coupling collar

A coupling collar 210 couples the cap 114 to the stand 112 and holds them in place during the procedure. The coupling collar 210 generally has a ring shape and fits around the pedestal support portion 200 of the cap 114. The coupling collar 210 includes bearing surfaces 212 and 214 that engage the cap 114, centering the coupling collar 210 on the cap 114 and engaging and clamping the flange portion 194 of the cap 114 to the stand 112. The coupling collar 210 also includes coupling arms 216 extending generally parallel to the longitudinal axis 140 of the stand 114 about a circumference of the coupling section 150 of the stand 112, either as a circumferentially continuous flange or as one or more discrete arms 216, as shown in the illustrated embodiment. The coupling arms 216 define inwardly-facing helical channels 218 or protrusions/screw threads that mate with and interlock the corresponding helical channels 156 or screw threads on the coupling section 150 of the stand 112. The coupling collar 210 is rotatable between a first position where the cap 114 is held to the stand and a second position rotatably removed from the first position where the cap 114 is free to move axially relative to the stand 112. The coupling collar 210 also includes outwardly-extending finger grips 219 that help the technician rotate the coupling collar 210 between the first position and the second position.

Rotating the cap 114 relative to the stand 112 could displace the donor corneo-scleral button, making it difficult or impossible to prepare a corneal graft from a desired portion of the corneo-scleral button. The cap 114 is mounted over the stand 112 axially, typically without rotation. Consequently, the illustrated cap 114 and stand 112 provide sufficient clearance between the inner surface of the cap 114 and the outer surface of the stand 112 to minimize or eliminate any contact between the inside surface of the cap 114 and the button as the cap 114 is placed on the stand 112. The inwardly stepped distal portion 172 of the pedestal 170 not only helps to define the cavity in which the corneo-scleral button is received, but also helps to space the inside surface of the cap 114 from the corneo-scleral button until after the corresponding anti-rotation elements engage, e.g., after the holes 198 in the registration section 196 of the cap 114 receive the ends of the pins 162 on the stand 112.

When engaged, the anti-rotation elements keep the cap 114 from rotating as it axially engages the corneo-scleral button and the coupling collar 210 secures the cap 114 to the stand 112. The anti-rotation pins 162 also can function as pilots to guide the cap 114 to move axially without tilting to one side or another as it is placed over the stand 112.

As the cap 114 is placed over the stand 112, the pins 162 enter the corresponding holes 198 in the registration section 196 of the cap 114 before the inside surface of the cap 114 engages the corneo-scleral button. Specifically, when the opening to a hole 198 is even with the top of a pin 162 the distance between the frustroconical surface of the cap 114 and the corresponding surface of the stand 112 is at least about the approximate thickness of a corneo-scleral button, typically about 550 microns. Preferably, there is zero clearance between the frustroconical surface of the cap 114 and the corresponding surface of the stand 112 when the cap 114 is mounted on the stand 112 without a corneo-scleral button therebetween. Variations in scleral thickness can be accommodated without any changes to the artificial chamber. The sclera can be compressed to hold the disk in place. A thicker sclera may be clamped more tightly than a thinner sclera without any change to the artificial chamber or its operation.

### Keratome

The illustrated cap 114 also includes a keratome support 220 offset from one side of the support section 200 of the cap 114, extending radially outward relative to the longitudinal axis of the cap 114. The keratome support 220 in the illustrated embodiment takes the form of a hollow box, the top of which is even with the top 122 of the cap 114 to provide additional support for the base 132 of the keratome 130.

The keratome 130 is mountable to the artificial chamber 110, particularly to the top of the cap 114, and can help define the top end 122 of the cap 114. The base 132 can be adhered to the cap 114, welded thereto, or integrally formed with the cap 114. Additionally or alternatively, a clip 224 may be provided to hold the keratome 130 to the cap 114.

### Clip

In the embodiment shown in FIGS. 2-5, a clip 224 holds the keratome 130 to the cap 114. The cap 114 has a cylindrical slot 226 in a top end 122 in which a cylindrical mounting flange 230 depending from the base 132 of the keratome 130 is received. When the cap 114 is mounted over the stand 112, the mounting flange 230 and the corresponding slot 226 in the top of the cap 114 also align the opening 134 in the base 132 of the keratome 130 with the top of the pedestal portion 170 of the stand 112 and the opening 120 in the cap 114.

The cap 114 also includes laterally-spaced arms 232 extending upward from the top end 122 of the cap 114, generally parallel to the longitudinal axis of the cap 114. Distal portions 234 of the arms 232 turn inwardly. The inwardly-turned portions 234 of the arms 232 are spaced from the top end 122 of the cap 114 sufficiently for the base 132 of the keratome 130 and the mounting flange 230 to fit therebetween so that the mounting flange 230 can be aligned with and moved into the slot 226 in the top of the cap 114.

The clip 224 fills the resulting gap between the inwardly-turned portions 234 of the arms 232 and the base 132 to hold the base 132 of the keratome 130 to the top of the cap 114. The clip 224 includes a grip portion 314 and laterally spaced tines or fingers 316 extending from the grip portion 314.

The clip 224 is transversely movable relative to the longitudinal axis of the cap 114 and the fingers 316 substantially fill the space between the top of the base 132 and the inwardly-extending portion 234 of the arms 232, thereby holding the keratome 130 to the cap 114.

### Integral Keratome

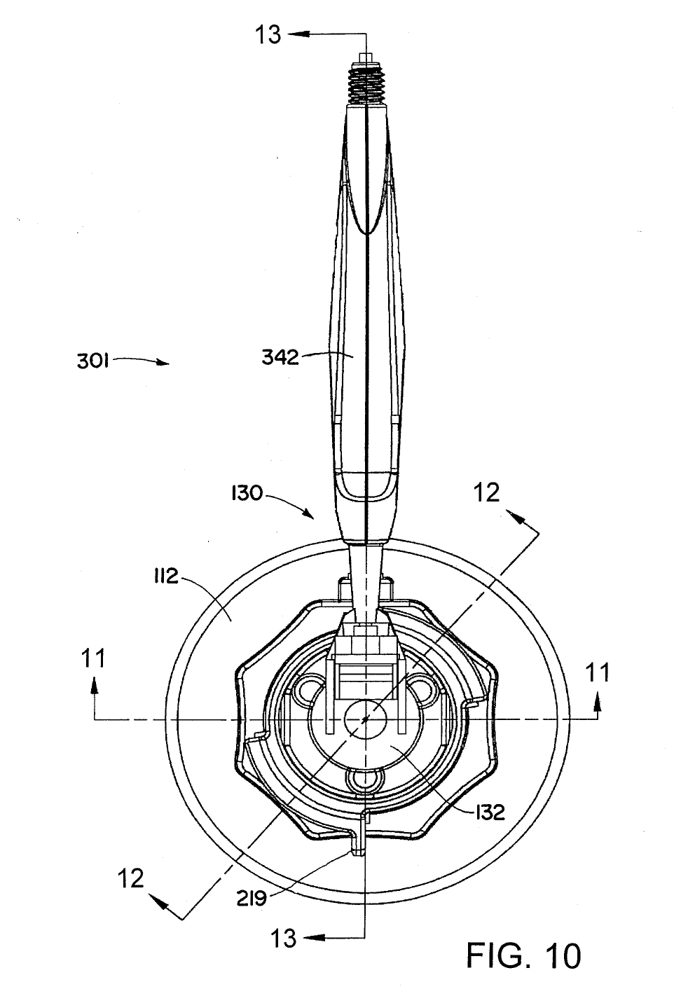

An alternative embodiment of the combination 109 is shown in FIGS. 6-13. The illustrated combination 301 is substantially the same as the combination 109 of FIGS. 2-5, and similar elements are identified by the same reference numbers. In this combination 301, however, the base 132 of the keratome 130 is an integral part of the cap 302, obviating the need for the clip 224. The base 132 and the cap 302 may be molded as a single part, or as in the illustrated embodiment, the base 132 and the cap 302 can be formed separately and then attached to each other, such as with an adhesive, by welding or otherwise bonding the parts together.

As in the previous embodiment, the cap 302 has a cylindrical slot 226 in a top end 122 of the cap 302 in which a cylindrical mounting flange 230 depending from the base 132 of the keratome 130 is received. In this embodiment, the top of the base 132 defines the top of the cap 302. The slot 226 and the flange 230 provide additional surface area for bonding the base 132 to the cap 302. The stand 112, the coupling collar 210, the keratome 130 and most of the cap 302 are substantially identical to the corresponding structures in the previous embodiment, omitting primarily the arm extensions 232 on the cap 114 and the clip 224 for holding the keratome 130 to the cap 114 (FIG. 2). In all other respects the combination 301 shown in FIGS. 6-13 is identical to that of FIGS. 2-5 and common reference numbers are employed to reference common elements.

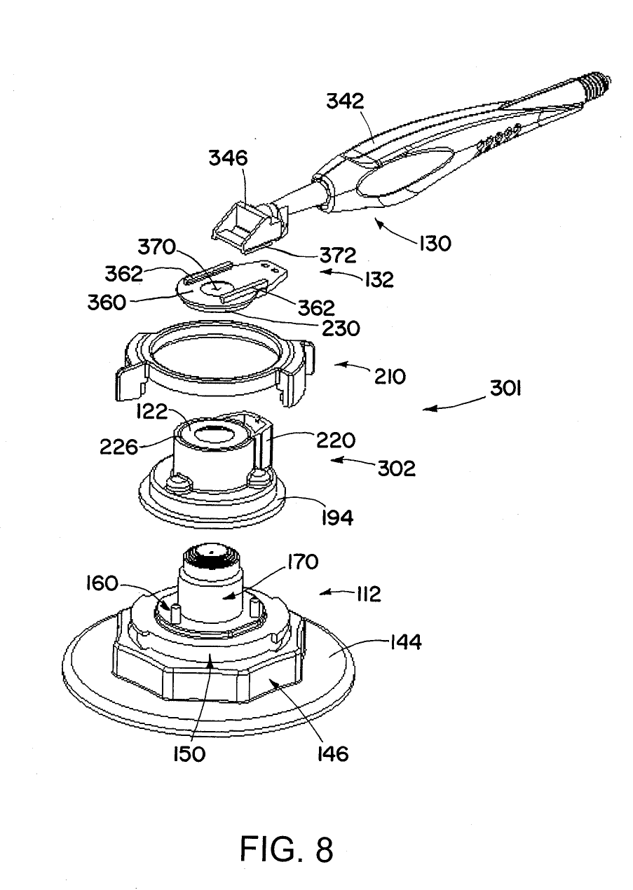

In some instances the artificial chamber can be used without the base 132. With just the cap 302 mounted over the stand 112 and secured in place by the coupling collar 210, the corneo-scleral button can be cut with a laser, for example, or otherwise clamed in the artificial chamber without being cut by a keratome and thus without the need for the base 132 to be attached to or formed as part of the cap 302. In this configuration, the top 122 of the cap 302 shown in FIG. 8 can act as a keratome base for registration of a laser without the keratome base 132 and accompanying carriage and cutting blade.

### Handle

Returning to a further description of the keratome 130, with reference to FIGS. 2-15, the base 132 includes an extension at one end that synergistically provides a handle 342 for holding the keratome 130. The technician can use the handle 342 to hold the keratome 130 during the preparation procedure. Consequently, the handle 342 generally must extend a sufficient distance for an adult to place two fingers and a thumb on the handle 342, and more typically extends at least about 2 inches (about 5 cm), at least about 3 inches (about 7.6 cm), at least about 4 inches (about 10 cm) or more.

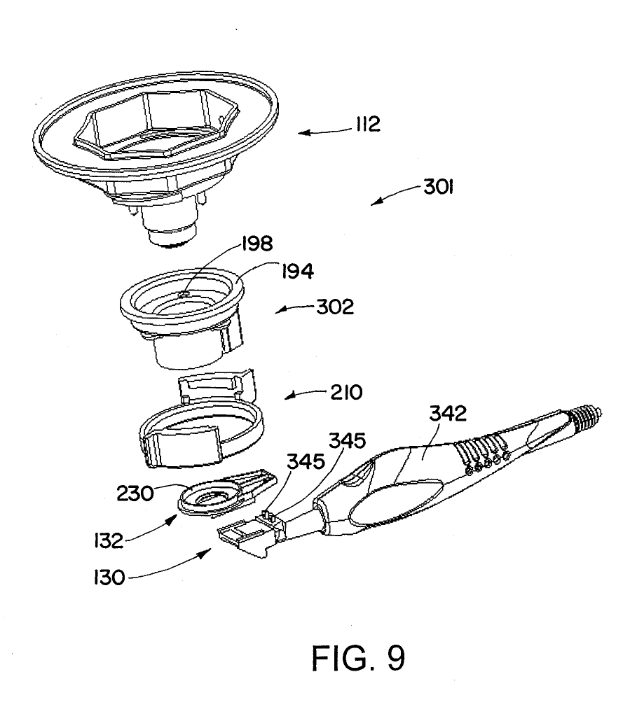

The base 132 has at a back end thereof one or more holes or openings 344 for fixing the handle 342 thereto by suitable means. Any suitable method of attachment may be used, including the illustrated tabs 345 (FIG. 9) which snap into position when pressed into the corresponding openings 344 in the base 132, thereby providing a snap-fit connection between the handle 342 and the base 132.

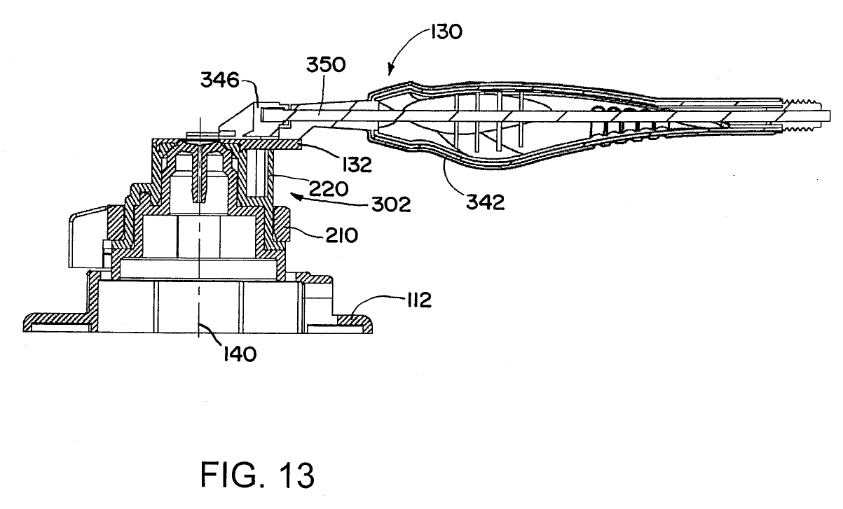

In addition to the base 132, the keratome 130 has a carriage 346 mounted to the base 132 that carries the cutting blade 136. The handle 342 holds and guides a drive shaft 350 (FIG. 13) that is connected to the carriage 346 for movement therewith. The carriage 346 supports and guides the cutting blade 136. The handle 342 is disposable as part of the keratome 130, and can be preassembled, pre-sterilized and then connected to the base 132 and the carriage 346. Alternatively, the handle 342 can be cast or molded as a single piece, and may be formed as a unitary part of the base 132, which itself may be formed as a unitary part of the cap 114. Thus the base 132 may be integral with either the cap 114 or the handle 342 or both.

### Base

The base 132 has a substantially flat top surface 360 on which the carriage 346 rests. The base 132 provides a registration face for guiding the cutting blade 136. The carriage 346 is guided for movement in a cutting direction relative to the base by one or more guide members. Extending from the top surface 360, the base 132 includes a pair of spaced apart parallel guides 362. The illustrated guides 362 have an inverted L-shape cross-section and oppose each other to cooperatively form a track or guideway. The guides 362 cooperate with the carriage 346 to restrain and guide the carriage 346 for linear movement along the base 132 in a cutting direction parallel to the length of the guides 362. The guides 362 also hold the carriage 346 to the top surface 360 of the base 132 against any significant separation therefrom, thereby maintaining the carriage 346 in stable sliding engagement with the top surface 362 of the base 132.

Toward a front end of the base 132 opposite the handle 342 and between the guides 362, a generally circular opening 370 is provided for receiving a cornea therethrough. If the base 132 is integral with the cap 114, the opening 370 also is the opening 120 in the cap 114. The opening 370 communicates through the base 132 to the cavity formed by the stand 112 and the cap 114. Different caps 114 and/or different bases 132 having different size openings 120 and/or 170 can be provided to accommodate different size corneas or corneal disks. The carriage 346 is guided for movement across the opening 370 in the base 132.

### Carriage

The carriage 346 is substantially similar to the cutting assembly disclosed in International Patent Publication No. WO 0217834 A2, published in the English language on 7 Mar. 2002, which is hereby incorporated herein by reference in its entirety. A pair of parallel guide rails 372 extend from the sides of the carriage 346 to cooperatively engage the guides 362 on the base 132.

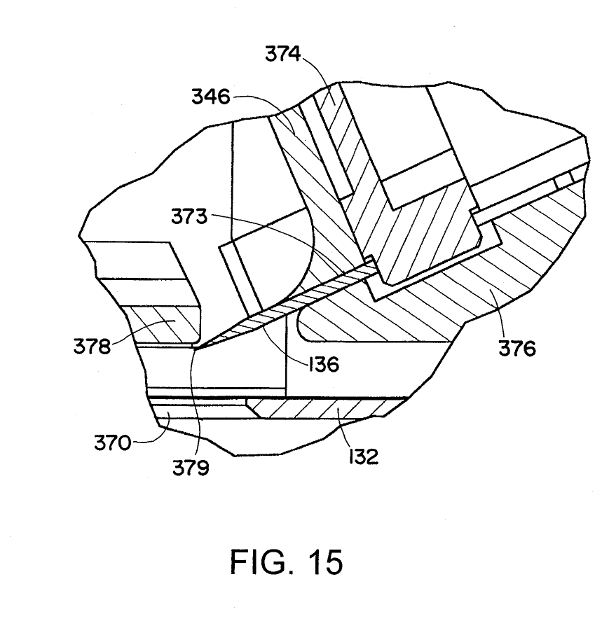

Referring to FIG. 8, the carriage 346 has formed therein a substantially vertical slot that opens from an angled blade guide surface 373. The slot slidingly receives and guides a blade holder 374 (FIG. 15) for transverse reciprocating movement therein. The width of the slot is transverse to the direction of the motion of the carriage 346 and is wider in the transverse direction than the blade holder 374 to permit the blade holder 374 to oscillate within the slot. The transverse oscillation of the blade holder 374 creates a transverse oscillation of the cutting blade 136 which is connected to the blade holder 374 as described below.

The blade holder 374 fits through an opening in the cutting blade 136 to engage and move the cutting blade 136. The blade holder 374 has a vertical slot which is perpendicular to the width of the blade holder 374. When the blade holder 374 is assembled in the carriage 346 the slot is aligned with a substantially horizontal opening or passage. The passage generally is perpendicular to the slot in the blade holder 374.

The cutting blade 136 is held between the angled blade guide surface 373 and a parallel angled top surface of a wedge 376. The wedge 376 supports the cutting blade 136 and the blade holder 374 in the carriage 346. The wedge 376 has an approximately triangular cross-section with a thinner portion facing forward. The angled top surface supports the cutting blade 136 as it oscillates with the blade holder 374.

Forward of the leading edge of the cutting blade 136, in the cutting direction, the carriage 346 includes a sled 378 with a bottom surface above the leading edge of the cutting blade 136. The sled 378 forms a generally horizontal shelf that is interposed between the guide rails 372 extending from the sides of the carriage 346. The rails 372 are parallel to and slidingly engage the guides 362 on the base 132. As the carriage 346 moves across the opening 370 in the base 132, a bottom surface of the sled 378 engages and flattens the surface of the cornea in preparation for cutting by the trailing cutting blade 136.

The distance between the lower surface of the sled 378 and the leading edge 379 of the cutting blade 136 defines the maximum thickness of the corneal flap or resection. Variations in this distance, provided by different carriages with different cutting blade-to-sled distances (cutting depth), provide the ability to obtain a corneal resection of the desired size and thickness. The keratome 130 with a selected cutting depth generally is selected based on the size of the donor cornea.

The carriage 346 (except the cutting blade 136) and the base 132 (although not necessarily the handle portion 342) may be composed of transparent materials, such as a transparent molded plastic, to maximize the technician's view of the procedure. To provide a high quality cut, the top surface 360 of the base 132 must be flat to within about one-thousandth of an inch (about 0.0254 millimeters). With advanced molding technology this can be achieved in a disposable material, such as plastic. In contrast, many existing keratomes are made of stainless steel. The process for machining metal to that degree of accuracy makes metal keratomes much more expensive, however, which economically requires them to be reused rather than disposable.

The cleaning and sterilization necessary for reuse is difficult, laborious, and time consuming, often taking from two to four hours to complete and requiring a trained technician. Moreover, if this process is performed at an eye bank in the United States, the procedure is regulated by the U.S. Food and Drug Administration (FDA), which requires yearly certification. The procedure also may be regulated by other governmental agencies in other countries. All of these factors make a metal keratome or a metal artificial chamber even more expensive to use. For these reasons a disposable keratome is preferred, particularly a substantially plastic keratome.

### Connection

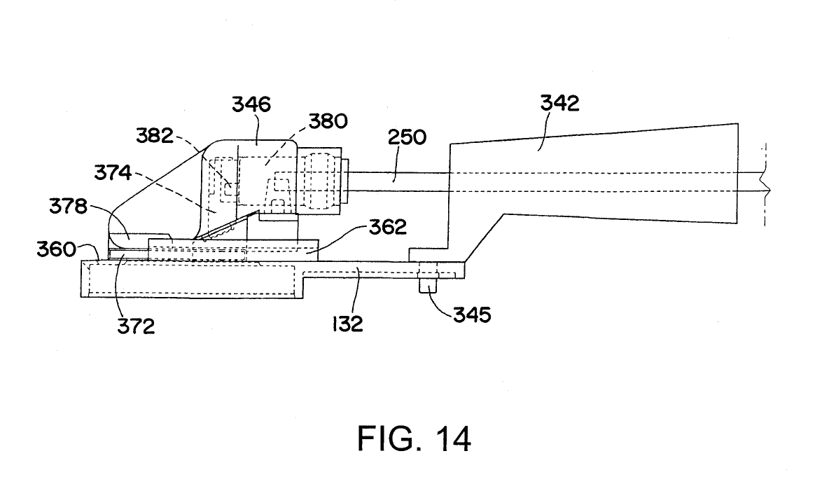

At a forward end of the handle 342, the drive shaft 350 (FIG. 14) is connected to the carriage 346. The drive shaft 350 has a fitting 380 on a proximal end thereof, from which an eccentric 382 extends. The fitting 380 passes through the horizontal passage in the carriage 346 to extend the eccentric 382 into engagement with the vertical slot in the blade holder 374. The fitting 380 can be press fit into the passage until it snaps or locks into the annular recess, thereby securing the fitting 380 in the carriage 346 such that the fitting 380 can freely rotate while providing a positive connection between the drive shaft 350 and the carriage 346 for both advancing and retracting the carriage 346 along the base 132.

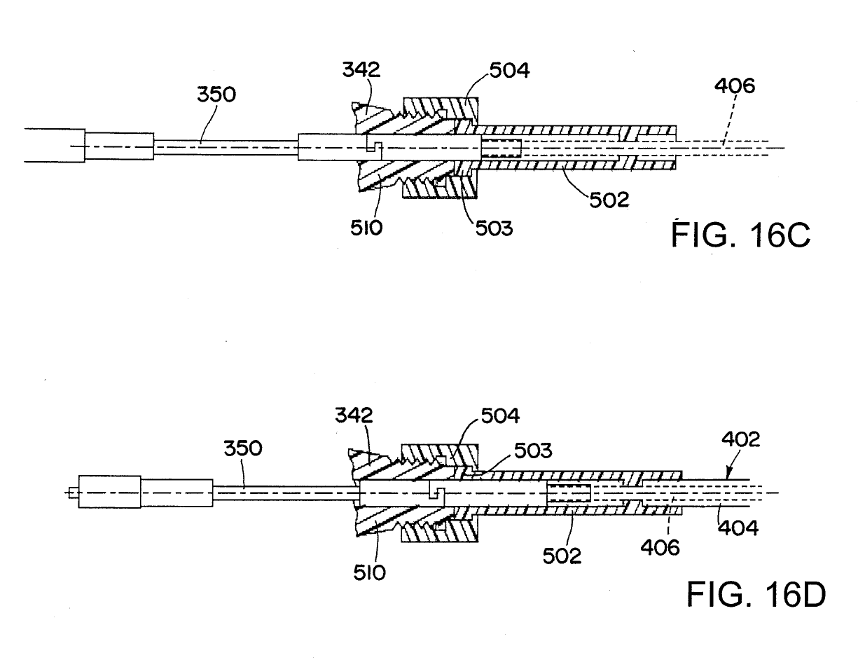

The keratome 130 generally, and particularly the base 132, also is configured for connection to the control assembly 106 (FIG. 1). An exemplary control assembly 400 is shown in FIG. 17 and includes a control cable 402 having a sheath 404 and a control shaft 406 movable within the sheath 404. The keratome 130 includes an anchor for fixing the sheath 404 relative to the base 132 such that axial movement of the control shaft 406 effects movement of the carriage 346 in the cutting direction. More particularly, the distal or terminal end of the handle 342 acts as the anchor and is connectable to the control cable 402.

The control cable 402 is connected to the rear of the handle 342, such as with a quick-connect coupling, so that the sheath 404 is removably fixed and generally held stationary relative to the handle 342, and thus to the base 132, and the control shaft 402 is connected to the drive shaft 350 in the handle 342. Any means for connecting the control shaft 406 to the drive shaft 350 is acceptable, as long as it provides a positive connection for the transmission of rotational motion and both forward and reverse axial motion. The drive shaft 350 transmits both the rotational and axial motion of a control shaft 406 from the control cable 402 to the carriage 346 and the cutting blade 136.

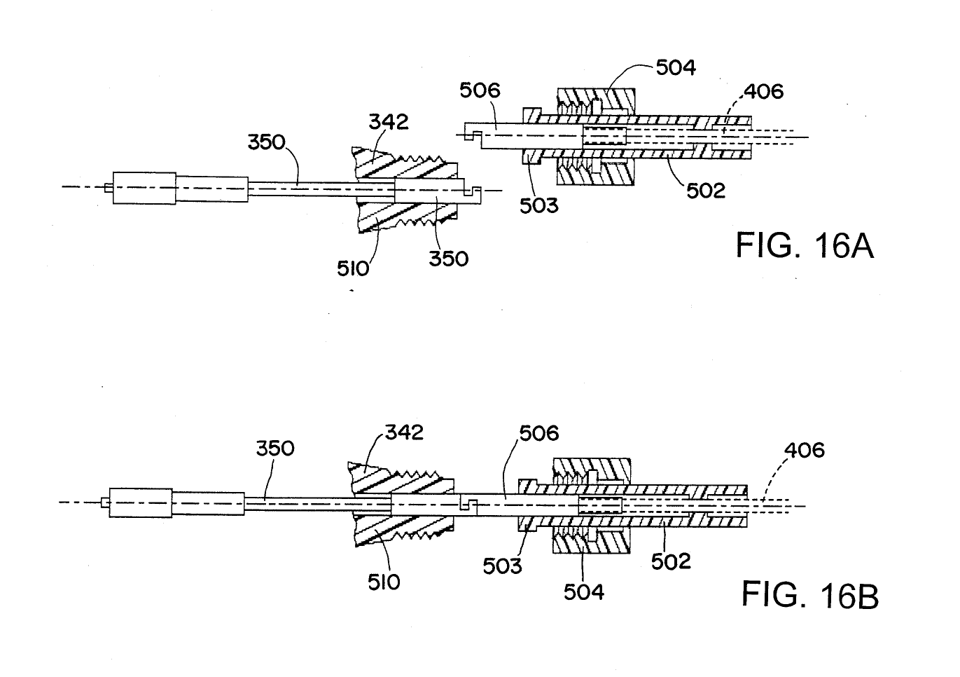

One type of connection between the keratome 130 and the control cable 402 is shown schematically in FIGS. 16A-16D, for example. In these figures, the sheath 404 of the control cable 402 is swaged into a cylindrical fitting 502 through which the control shaft 406 extends. The distal end of the cylindrical fitting 502 has an annular flange 503 which retains a threaded nut 504 which fits over the cylindrical fitting 502 and the sheath 404. The nut 504 is retracted, as shown in FIGS. 16A and 16B, for the connection of a control shaft fitting 506 to the drive shaft 350. The drive shaft 350 protrudes from the handle 342 and has a shape which partially overlaps and mates with the fitting 506 on the control shaft 406.

The control shaft 406 can be extended to push the drive shaft 350 into the handle 342 and to draw the sheath 402 toward the handle 342.

A portion 510 of the rear end of the handle 342 also is threaded, or includes a threaded part mounted thereto, which mates with the threaded nut 504 on the control cable 402. Once the control shaft 406 and the drive shaft 350 are connected, the control shaft 406 is advanced to move the nut 504 into engagement with the rear end of the handle 342, and the threaded parts 510 and 504 are screwed together, as shown in FIG. 16C, to secure the sheath 404 to the handle 342 and to lock the shafts 350 and 406 together. The control shaft 406 can then be retracted to positively retract the drive shaft 350 along with it, as shown in FIG. 16D.

The rotational motion of the control shaft 406 is transmitted to the carriage 346 by the drive shaft 350 where the components of the carriage 346 cooperate to transform the rotational motion into an oscillating motion for driving the cutting blade 136 in an oscillating side-to-side motion. Linearly advancing and retracting the control shaft 406 in the sheath 404 effects corresponding movement in the carriage 346 independent of the rotation.

### Control Assembly

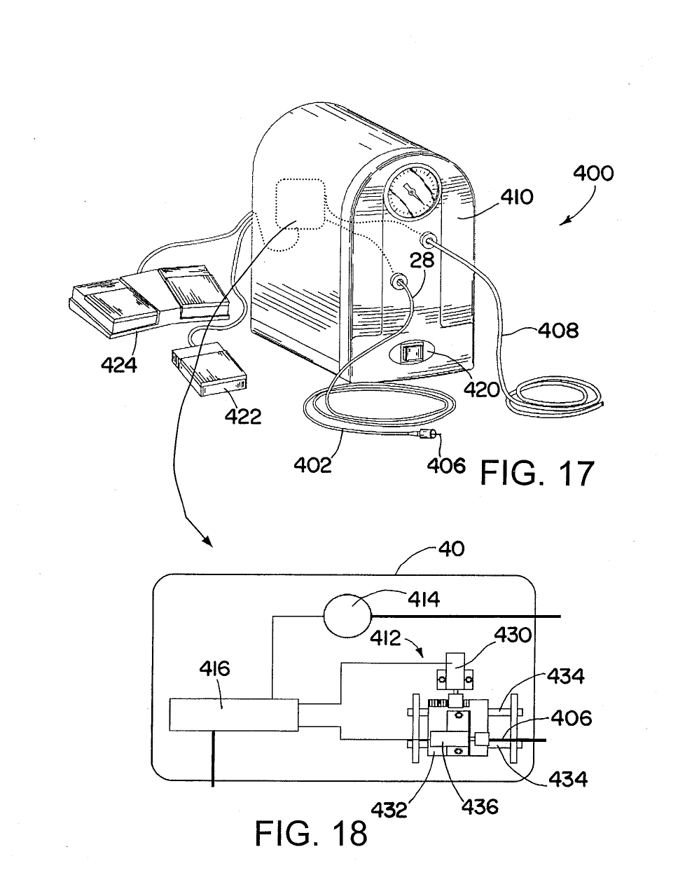

The control assembly 400 shown in FIGS. 17 and 18 controls and drives the control shaft 406. The illustrated control assembly 400 controls the axial and rotational movement of the control shaft 406, and the supply of fluid to an artificial chamber 102 (FIG. 1). The control assembly 400 includes a flexible fluid line or tube 408 and at least one (preferably only one) control cable 402. The control cable 402 connects the control assembly 400 to the keratome 130 (FIG. 2) for remotely controlling and driving the keratome, particularly the cutting blade 136 via the carriage 346 and drive shaft 350 (FIG. 13).

The control assembly 400 is substantially contained within a housing 410 and includes a drive assembly 412, a fluid pump 414 and a controller 416. The controller 416 can include an electronic circuit for controlling the operation of the drive assembly 412 and the pump 414. An exemplary pump 414 is a pneumatic pump or compressor that supplies pressurized air through the tube 408 to the fitting 184 in the base of the artificial chamber and the fluid channel 182 (FIG. 11). The tube 182 can include an in-line filter (not shown) that is only used once before being replaced, along with the tube 182, for each new artificial chamber.

The control assembly 400 also has one or more input devices connected to the controller 416, such as a touch screen, an ON/OFF switch 420, a fluid control pedal or switch 422, a two-position drive pedal or switch 424, etc. The one or more input devices permit the technician to control several variables in the operation of the system, as further described herein.

The drive assembly 412 is substantially similar to the drive assembly disclosed in the aforementioned International Publication. The drive assembly 412 includes a linear drive motor 430 connected to a slide member 432 by a rack and pinion, for example, for moving the slide member 432 through a range of linear motion corresponding to the desired cut length. The slide member 432 is mounted on one or more slide guides 434 for directing or guiding the linear motion of the slide member 432. A rotary drive motor 436 is carried on the slide member 432 for movement therewith. The control shaft 406 inside the control cable 404 is connected to the rotary motor 436. Because the rotary drive motor 436 is mounted on the slide member 432, the drive shaft 406 can simultaneously or independently transfer rotational motion (created by the rotary drive motor 436) and linear axial motion (created by the linear drive motor 430) to the keratome.

### Method

To begin the procedure, the technician selects the optimum keratome for the size of the donor cornea and the desired depth of cut to provide the desired size corneal resection. The keratome comes preassembled and pre-sterilized and thus requires no assembly or sterilization. The technician removes the packaging and connects the keratome to the control assembly. Alternatively, the technician may have to mount the carriage or the handle to the base of the keratome or the base of the keratome to the cap. The technician generally double-gloves to maintain sterility, removing one set of gloves after touching reusable components of the control assembly. A new, and sterile, fluid tube is connected to the fixture in the stand or to a section of tubing extending therefrom and to the fluid pump, whether separate or incorporated into the control assembly. Alternatively, a fluid-filled syringe can be substituted for the pump. The control cable is connected to the handle of the keratome so that the sheath is anchored to the base and the control shaft engages the drive shaft. The technician or surgeon can then hold the keratome by the handle without handling any other component of the system.

The donor corneo-scleral button is placed on the pedestal portion of the stand and the cap is mounted axially onto the stand. The anti-rotation elements engage before the cap engages the corneo-scleral button to guide the cap onto the stand, thereby minimizing or eliminating any rotation of the cap that would move the corneo-scleral button out of position.

Then the coupling collar is rotated to engage the threads or helical recesses on the stand and secure the cap on the stand. The keratome, if not already mounted to the cap, can be secured to the top of cap. Finally, the technician turns on the pump to provide fluid through the channel to support the back side of the corneo-scleral button and push the cornea through the opening in the base of the keratome.

Operation of the keratome is substantially automatic and proceeds in substantially the same manner as the operation of the keratome described in the aforementioned International Publication. The technician can control the keratome with foot pedals, without touching the control assembly with his hands, further maintaining sterility. The cutting blade is driven across the opening to cut a corneal flap or resection from the donor corneo-scleral button. Once the procedure is complete, the keratome can be disconnected from the control assembly and discarded along with the cap and stand that make up the artificial chamber. The flap or resection cut from the button is replaced until the surgeon is ready to cut the disk from the button for implantation. The button also can be left in the cavity between the cap and stand until the surgeon is ready to cut the disk from the button.

A new keratome and artificial chamber are selected for a subsequent procedure and another corneo-scleral button can be mounted in the artificial chamber in a matter of minutes, essentially eliminating the down time for cleaning and sterilizing a reusable keratome and artificial chamber. Current cleaning and sterilizing procedures can take hours, severely limiting the number of procedures that can be performed in a day. This is particularly troublesome since corneo-scleral buttons can only be conserved in fluid for a few days. In addition a specially trained technician is required to perform the cleaning and sterilization.

The control assembly includes components that are expensive to produce and are intended to be reused for surgery on many patients, whereas the keratome and artificial chamber include relatively inexpensive components and are intended to be used for a single cornea and then discarded. Because the control assembly is removed from the surgical area, contamination of the control assembly by the corneal tissues, and vice versa, is minimized or prevented. Maintaining a high degree of confidence in the sterility of a keratome and the artificial chamber has been a problem for which the present invention provides an improved solution.

Although the invention has been shown and described with respect to certain illustrated embodiments, equivalent alterations and modifications will occur to others skilled in the art upon reading and understanding the specification and the annexed drawings. In particular regard to the various functions performed by the above described integers (components, assemblies, devices, compositions, etc.), the terms (including a reference to a “means”) used to describe such integers are intended to correspond, unless otherwise indicated, to any integer which performs the specified function (i.e., that is functionally equivalent), even though not structurally equivalent to the disclosed structure which performs the function in the herein illustrated embodiments of the invention. In addition, while a particular feature of the invention may have been described above with respect to only one of several illustrated embodiments, such a feature may be combined with one or more other features of the other embodiment, as may be desired and advantageous for any given or particular application.

1. An artificial anterior chamber for preparing a corneal graft from a corneo-scleral button, comprising:
(A) a stand having a support pedestal to support a donor corneo-scleral button at a distal end;
(B) a cap mountable over the support pedestal; and
(C) a coupling collar for securing the cap to the base without rotating the cap relative to the base.

2. An artificial chamber as set forth in claim 1, wherein the coupling collar and at least one of the stand and the cap include mating elements that can engage to clamp the cap to the stand.

3. A system as set forth in claim 1, wherein the coupling collar has a bearing surface that engages an outwardly-extending flange portion of the cap and a coupling element that engages a corresponding coupling element on the stand to pull the bearing surface against the flange, thereby clamping the cap to the stand.

4. A system as set forth in claim 3, wherein the coupling collar is rotatable between a first position holding the cap to the stand and a second position angularly offset from the first position to allow cap to be removed from the stand.

5. An artificial chamber as set forth in claim 1, wherein the coupling collar substantially encircles the support portion of the cap.

6. A system as set forth in claim 1, wherein the stand includes a coupling portion having outwardly-facing screw threads.

7. A system as set forth in claim 6, wherein the coupling collar has a bearing portion for engaging a flange portion of the cap and a coupling portion with inwardly-facing screw threads for engaging the outwardly-facing screw threads on the stand, whereby turning the coupling collar to engage the corresponding threads pulls the bearing portion against the flange to clamp the cap against the stand.

8. A system as set forth in claim 7, wherein the coupling collar is rotatable between a first position holding the cap to the stand and a second position angularly offset from the first position to allow cap to be removed from the stand.

9. A system as set forth in claim 1, wherein the stand, the cap, and the coupling collar are plastic.

10. A system as set forth in claim 1, wherein the pedestal portion of the stand is fixed relative to the rest of the stand.

11. An artificial chamber as set forth in claim 1, wherein the coupling collar is mounted over the cap for rotation relative to the cap.

12. An artificial chamber as set forth in claim 1, wherein the cap has a support portion that fits over at least a portion of the support pedestal, and an outwardly-extending flange toward a bottom end of the support portion.

13. An artificial chamber as set forth in claim 12, wherein the cap includes a keratome base toward a top end of the support portion that extends beyond a laterally outer extent of the support portion.

14. An artificial anterior chamber for preparing a corneal graft from a corneo-scleral button, comprising:
(A) a stand having a support pedestal to support a donor corneo-scleral button at a distal end; and
(B) a cap mountable over the distal end of the support pedestal;
wherein the stand and the cap include cooperating anti-rotation elements that prevent relative rotation between the cap and the stand.

15. An artificial chamber as set forth in claim 14, wherein the anti-rotation elements include one or more pins and corresponding holes that receive the pins.

16. An artificial chamber as set forth in claim 15, wherein the stand includes one or more axial pins parallel to and spaced from a longitudinal axis of the pedestal, and one or more corresponding holes in a portion of the cap that receives the pins.

17. An artificial chamber as set forth in claim 14, wherein when the cap is placed over the stand and the corresponding anti-rotation elements begin to engage, an upper inside surface of the cap is spaced from an upper surface of the pedestal by at least 550 microns.

18. An artificial anterior chamber for preparing a corneal graft from a corneo-scleral button, comprising:
(A) a stand having a support pedestal to support a donor corneo-scleral button at a distal end; and
(B) a cap mountable over the distal end of the support pedestal;
wherein the stand and the cap include opposed generally parallel surfaces that cooperate to clamp a portion of the corneo-scleral button therebetween.

19-21. (canceled)

22. An artificial anterior chamber for preparing a corneal graft from a corneo-scleral button, comprising:
(A) a stand having a support pedestal to support a donor corneo-scleral button at a distal end, and a fluid channel that terminates at a top of the pedestal to provide fluid support for a corneo-scleral button mounted thereon; and
(B) a cap mountable over the distal end of the support pedestal so that an opening in a top of the cap provides access to a corneo-scleral button supported on the pedestal.

23. (canceled)

24. (canceled)

25. (canceled)

26. A system for preparing a corneal graft, comprising a sterile, disposable artificial anterior chamber for holding a corneo-scleral button; and a sterile, disposable keratome having a cutting blade for cutting a corneal portion of the button.

27. A system as set forth in claim 26, comprising a remotely located, reusable control assembly connectable to the keratome to control operation of the keratome.

28. (canceled)

29. (canceled)

30. (canceled)

31. A system as set forth in claim 26, wherein the artificial chamber includes a stand having a support pedestal to support a donor corneo-scleral button at a distal end, and a cap mountable over the distal end of the pedestal.

32. A system as set forth in claim 31, wherein the keratome includes a base and a carriage mountable to the base; the cutting blade is carried in the carriage, the carriage is guided for movement in a cutting direction relative to the base, and the base has an opening therein that can be aligned with the upper end of the pedestal to provide access to the donor cornea.

33. A system as set forth in claim 32, wherein the base defines the top of the cap.

34. A system as set forth in claim 32, wherein the keratome base is an integral part of the cap.

35-42. (canceled)

43. A system as set forth in claim 31, wherein a top surface of the keratome base is substantially flat to within about one thousandth of an inch (approximately 0.0254 millimeter).

44. A system as set forth in claim 26, wherein the keratome is substantially made of plastic.

45-54. (canceled)

55. A system as set forth in claim 26, comprising a clip that couples the keratome to the artificial chamber.

56. (canceled)

57. (canceled)

58. A system as set forth in claim 55, wherein the coupling clip is configured to transversely engage the base of the keratome and the cap of the artificial chamber to hold them together.

59. A method of preparing a corneal graft from a corneo-scleral button, comprising the steps of (A) mounting a corneo-scleral button in an artificial anterior chamber; (B) connecting a keratome to a remotely located control assembly; (C) controlling the keratome to cut a corneal portion of the button held in the artificial chamber; (D) disconnecting the keratome from the controller; and (E) disposing of the artificial chamber and the keratome.

60-74. (canceled)
